(19) United States
(12) Patent Application Publication
(10) Pub. No.: US 2008/0299082 A1
(43) Pub. Date: Dec. 4, 2008

(54) Novel recombinant T4 phage particle containing HIV, H. pylori or cancer antigens and uses thereof

(76) Inventors: Zhao-jun Ren, Somerset, NJ (US); Derhsing Lai, Yardley, PA (US)

Correspondence Address: CHERYL H AGRIS PHD, PO BOX 806, PELHAM, NY, 10803, US

(21) Appl. No.: 11/656,792

(22) Filed: Jan. 23, 2007

Related U.S. Application Data
(63) Continuation-in-part of application No. 11/188,236, filed on Jul. 22, 2005.
Application No. 11/188,236 is a continuation-in-part of application No. PCT/US05/26360, filed on Jul. 23, 2005.
(60) Provisional application No. 60/762,443, filed on Jan. 24, 2006.

(30) Foreign Application Priority Data
Aug. 4, 2004 (CN) 200410040389.9

(51) Int. Cl. A61K 35/76; C12N 7/00; C07K 14/00; C12N 15/11; C40B 40/02; A61P 31/00; C40B 30/04; C12N 15/00; C12N 1/20; C12P 21/04

(52) U.S. Cl. 424/93.2; 435/235.1; 530/350; 536/23.72; 435/320.1; 435/252.3; 435/69.1; 506/14; 506/9

(57) ABSTRACT

The invention is directed to a novel recombinant T4 phage particle expressing a HOC and/or SOC Helicobacter pylori and/or SCLC fusion peptide as well as methods for their preparation and methods of use in compositions and kits.

### PRIORITY CLAIM

This application claims priority under 35 U.S.C. §119(e) from application Ser. No. 60/762,443, the contents of which are incorporated herein by reference. This application is also a continuation in part of application Ser. No. 11/188,236, filed Jul. 22, 2005, the contents of which are incorporated herein by reference and a continuation-in-part of application no. PCT/US05/26360, filed Jul. 23, 2005, the contents of which are incorporated herein by reference. application Ser. Nos. 11/188,236 and PCT/US05/26360 claim priority under 35 USC 119(a)-(d) to Chinese application no. 200410040389.9, filed Aug. 4, 2004, the contents of which are also incorporated herein by reference.

### FIELD OF THE INVENTION

The invention is directed to a novel recombinant T4 phage particle expressing a HOC and/or SOC fusion peptide or protein as well as methods for their preparation and methods of use in compositions and kits.

### BACKGROUND OF THE INVENTION

A number of attempts have been made to express heterologous proteins in bacteriophages (reviewed in Adhya et al., 2005, Mol. Microbiol. 55: 1300-14). Filamentous bacteriophages M13 and fd have subsequently been extensively used to display proteins and short peptides on the minor capsid protein pIII (see, for example, Devlin et al., 1990, Science 249:404-406; Parmley and Smith., 1998, Gene 73:305-318; Perham et al., 1995, FEMS Microbiol. Rev. 17: 25-31; U.S. Pat. No. 6,420,113, U.S. Pat. No. 6,555,310, U.S. Pat. No. 6,057,098, Smith, 1985, Science 228:1315-1317) and major capsid protein pVIII (Greenwood et al., 1991, J. Mol. Biol. 220:821-827; Kang et al., 1991, Proc. Natl. Acad. Sci. USA 88:4363-4366). Icosahedral phage λ has also been used to display foreign proteins on the outer capsid protein gpD (Mikawa et al., 1996, J. Mol. Biol. 262: 21-30; Sternberg and Hoess, 1995, Proc. Natl. Acad. Sci. USA 92: 1609-1613) and on tail protein gpV (Dunn, 1995, J. Mol. Biol. 248:497-506; Maruyama et al., 1994, Proc. Natl. Acad. Sci. USA 91:8273-8277). Phage T7 can also display proteins on the capsid (O'Neil and Hoess, 1995, Curr. Opin. Struc. Biol. 5(4): 443-449; WO98/05344; U.S. Pat. No. 6,777,239 and Maruyama et al., 1994, Proc. Natl. Acad. Sci. USA 91:8273-8277).

However, there are significant limitations. For example, display of certain peptides is restricted when filamentous phage is used, or not possible, since the fused peptide has to be secreted through the E. coli membranes as part of the phage assembly apparatus. Since both pill and pVIII proteins are essential for phage assembly, it is difficult to display large domains or full-length proteins without interfering with their essential biological functions. In situations where large peptide sequences are displayed, their copy number per phage capsid is greatly reduced and unpredictable. Similar problems on the size and copy number have been encountered with the phage lambda display systems. It is often necessary to incorporate wild type protein molecules along with the recombinants to generate viable phage using either a helper phage or a partial genetic suppression of amber mutant (Hoess, 2002, Curr. Pharm. Biotechnol. 3:23-8).

### T4 Expression Systems

The phage T4 capsid contains three essential structural proteins: major capsid protein gp23 (930 copies per capsid), and two minor capsid proteins: gp24 (vertex protein, 55 copies) and gp20 (portal vertex protein 12 copies) (Black et al., 1994, Morphogenesis of the T4 head, p. 218-253. In J. D. Karam (ed), Bacteriophage T4.ASM Press, Washington, D.C.; Yanagide, 1997, J. Mol. Biol. 109:515-537; Fokine et al., 2004, Proc. Natl. Acad. Sci. (USA) 101, 6003-6008). In addition, the T4 virion outer surface is coated with two dispensable capsid proteins SOC (810 copies per capsid, molecular mass 9 kDa) and HOC (155 copies, 40 kDa). These proteins are regularly displayed on the T4 icosahedral lattice (Black et al., 1994, Morphogenesis of the T4 head, p. 218-253. In J. D. Karam (ed), Bacteriophage T4.ASM Press, Washington, D.C.; Yanagide, 1997, J. Mol. Biol. 109:515-537). SOC and HOC bind strongly to the capsid following capsid assembly and the capsid expansion triggered by DNA packaging that creates HOC and SOC capsid binding sites (Jiang et al., 1997, Infect. Immun. 65: 4770˜4777).

The use of T4 phage as a vector to express heterologous peptides or proteins has been explored (for early work see Casna and Shub, 1982, Gene 18:297-307). A variety of approaches have been attempted. Shub and Casna, 1985, Gene 37: 31-36, discloses the expression of a rIIB-lacZ gene fusion in T4 phage. JP62232384 discloses a dC-type recombinant T4 phage containing heterologous DNA as well as a method of expression in E. coli by simultaneous infection with the recombinant T4 phage and T4 phage having a normal gammaII gene. Singer and Gold, 1991, Gene 106:1-6, discloses a T4 expression system that contains the multiple cloning sites of pUC18/19 and T7 promoter and terminator. Hong et al., 1993, Gene 136:193-198 and Hong and Black, 1993, Virol. 194:481-490, discloses a T4 packaging system using the T4 non-essential capsid scaffold protein IPIII. Asimov et al., 1995, Virus Genes 10: 173-177, discloses construction of Homeric T4 displaying foreign peptides. WO00/06717 discloses a method for improving efficiency in phage display by modifying the coat protein.

Rao et al., 1992, Gene 113:25-33, discloses the use of a T4 packaging system to package limm434 DNA into proheads. Rao and Leffers, 1993, Virology 196:896-899 further discloses the construction of empty proheads and the use of these proheads as tools for expressing heterologous DNA.

Mullaney and Black, 1998, BioTechniques 25:1008-1012 discloses a T4 phage derived protein expression, packaging and processing system where an HIV-1 protease is fused to green fluorescent protein. The fusion protein is targeted within the phage with an N-terminal capsid targeting sequence.

Another approach has involved the display of heterologous proteins on T4 by their fusion to capsid proteins SOC or HOC. Jiang et al., 1997, Infection and Immunity 65:4770-4777, discloses the cloning of a 36 amino acid PorA peptide from Neisseria meningitides into T4 display vectors to generate fusions at the N terminus of HOC or SOC.

Ren et al., 1996, Protein Science 5:1833-1843 discloses C-terminal fusions of a tetrapeptide, the 43 residue V3 loop domain of HIV gp120, and poliovirus VP1 capsid protein (312 residues) to SOC. Ren et al., 1997, Gene 198:303-311 discloses construction of a T4 phage hoc gene display vector.

Ren and Black, 1998, Gene 215:439-444 and Steven et al., U.S. Pat. No. 7,041,441, discloses the display of full-length heterologous protein by fusion to SOC or HOC. The vectors contained either SOC or HOC. This article discloses the display of a 271 residue heavy and light chain fused IgG anti-egg while lysozyme to the COOH-terminus of the SOC capsid protein and HIV-1 CD4 receptor (183 amino acids) fused with the amino-terminus of HOC. Malys et al., 2002, J. Mol. Biol. 319:289-304, discloses a bipartite T4 phage display library containing SOC and HOC randomized peptide fusions displayed on the external capsid surface of T4. Specifically, SOC and HOC DNAs are attached to random five amino acid sequence peptide extensions to the C terminus of SOC via a tetra-alanine linker. The recombinant particle disclosed in both of these articles did not contain T4 IPII and IPIII proteins; it only contained the IPI and was e⁺, denV⁺ and alt⁺. SUMMARY OF THE INVENTION

The invention is directed to a recombinant T4 phage particle having or expressing one or more heterologous peptides, nucleic acids or genes, particularly a T4 HOC and/or SOC fusion peptide (also includes proteins). The term “peptide” as used herein encompasses peptide, polypeptide and protein sequences containing 4 or more amino acids and includes full length proteins.

The HOC or SOC protein or functional portion thereof is bound to one or more heterologous peptides. The heterologous peptide may, in a particular embodiment, contain up to about 1,150 amino acids and may encompass one or more proteins, HOC or SOC sequence. The heterologous peptide may be separated by a spacer or linker. In particular, the recombinant T4 phage particle contains or expresses one or more heterologous peptides such as a receptor, ligand, antigen, immunogen or toxin and antibody (e.g., IgG-Fab/Fv single or double chain). Examples of such heterologous peptides (including proteins) include, but are not limited to, a cancer antigen or immunogen, a tumor antigen, in particular, a small cell lung carcinoma (SCLC) antigen. The antigen or immunogen may be derived from a pathogen, in particular, an HIV antigen (e.g., gp160, variant gp41), an H. pylori antigen (see Permin and Andersen, 2005, Helicobacter 10 (suppl. 1):21-25 for examples of possible antigens); the sequences of H. pylori genome and deduced proteins are disclosed in Genbank accession no. AE001439. As defined herein, “derived from” means that the antigen or immunogen contains peptide sequences from the particular pathogen or tumor. In a related aspect, the invention is directed to a recombinant prokaryotic cell comprising the T4 phage particle of the present invention.

In a particular embodiment, the invention is directed to a recombinant T4 phage particle comprising a SOC and/or HOC fusion peptide, wherein said phage particle has inactive T4 endonuclease V and/or T4 lysozyme function and wherein said fusion peptide comprises SOC and/or HOC bound to one or more heterologous peptide(s) derived from a tumor antigen or immunogen, said antigen or immunogen selected from the group consisting of Cytokeratin 19 (CYFRA21-1) (sequence disclosed in Genbank Accession No. NM_{—}002276 and Bader et al., 1986, EMBO J. 5 (8), 1865-1875); neuron specific enolase (NEA) (sequence disclosed in Genbank Accession No. NM_{—}001579); carcinoembryonic antigen (CEA) (Genbank accession no. AF406955); C-reactive protein (Genbank accession no. M11880 and Woo et al., 1985, J. Biol. Chem. 260:13384-13388); and/or Pro-gastrin-releasing peptide (ProGRP) (Genbank accession no. K02054 and Spindel et al., 1984, Proc. Natl. Acad. Sci. USA 81:5699-5703), a heterologous peptide(s) derived from a Helicobacter pylori (H. pylori) antigen, immunogen or toxin selected from the group consisting of H. pylori urease (Genbank Accession No. M60398 and Labigne et al., 1991, J. Bacteriol. 173:1920-1931), HspA (heat shock protein A, disclosed in Genbank Accession No. DQ141574 and Xu et al., World J. Gastroenterol 11:114-7) and/or VacA (disclosed in Genbank Accession No. AE001439 and Cover et al., 2005, Nature Reviews Microbiol. 3:320:332) and/or a heterologous peptide derived from a human immunodeficiency virus (HIV) peptide selected from the group consisting of gp160 and/or variant gp41. In a specific embodiment the HIV peptide is a variant gp41 which is resistant to HIV fusion inhibitor such as enfuvirtide and BMS-788806. Such variants include but are not limited to V38A, Q40H, N43D, G36S+L44M, N42T+N43K, N42T+N43S, V38A+N42D, V38A+N42T and V38E+N42S.

These fusion peptides may be obtained from the T4 particle of the present invention. The invention further relates to a nucleic acid molecule encoding said fusion peptide as well as a nucleic acid construct, vector and prokaryotic cell comprising said nucleic acid molecule. In another related aspect, the nucleic acid molecule encoding the fusion peptide may comprise sequences encoding T4 IPII and IPIII or functional peptides thereof, but devoid of nucleic acid sequences encoding functional T4 lysozyme and/or endonuclease V.

In another embodiment, the recombinant T4 phage particle expresses a HOC and/or SOC fusion peptide and has inactive T4 endonuclease V and T4 lysozyme function which may alternatively be referred to as denV⁻ and e⁻ respectively. The recombinant T4 phage particle may further be ipII⁺ and ipIII⁺ or alternatively referred to as containing nucleic acid sequences encoding functional IPII and IPIII proteins.

The invention is further directed to methods for obtaining the recombinant phage particles of the present invention. In one embodiment, the method comprises (a) providing a vector comprising a nucleic acid sequence encoding HOC and/or SOC fusion peptide; (b) providing a recombinant hoc and/or soc negative or truncate expression system; (c) culturing a prokaryotic cell (e.g., E. coli) with the vector of (a) and expression system of (b) under conditions to obtain recombination of (a) and (b) and obtain said recombinant T4 particle and (d) isolating said recombinant T4 phage particle. In a related aspect, the invention is directed to a prokaryotic cell comprising said recombinant T4 phage particle.

In a related aspect, the invention is directed to a method for obtaining a HOC-fusion peptide (includes full length protein) by culturing a prokaryotic cell comprising said T4 phage particle under conditions suitable for the production of the peptide and isolating the peptide. In a further embodiment, the SOC and/or HOC-fusion peptide may contain a cleavage site. Therefore, the invention may further be directed to a method for obtaining a heterologous peptide comprising culturing a prokaryotic cell comprising the recombinant T4 phage of the present invention, isolating the SOC and/or HOC fusion heterologous peptide and cleaving the SOC and/or HOC sequence from the fusion heterologous peptide and isolating the heterologous peptide.

The invention is further directed to a hoc and/or soc negative (also referred to a hoc⁻ and/or soc⁻), and optionally ipII⁻ and ipIII⁻ T4 phage expression system. As defined herein, a T4 phage expression system is a system containing T4 expression control sequences. In a particular embodiment, the T4 phage expression system is e⁻, and/or denV³¹ . The invention is further directed to a method for obtaining the T4 expression system of the present invention which is hoc or soc negative, comprising the steps of: (a) providing a hoc⁻ or soc⁻ T4 phage; (b) providing a hoc⁻ or soc⁻, e⁻, and/or denV⁻ T4 phage particle, wherein (a) and (b) are both hoc⁻ and/or both soc⁻; (c) culturing a prokaryotic cell with (a) and (b) under conditions promoting recombination of (a) and (b) and (d) isolating said expression system. A hoc⁻ and soc⁻ negative expression system may be obtained by (a) providing a hoc⁻ recombinant T4 phage expression system; (b) providing a soc⁻ recombinant T4 phage expression system and (c) culturing (a) and (b) in a prokaryotic cell under conditions promoting recombination of (a) and (b) and (d) isolating said expression system.

The invention is further directed to a composition comprising one or more of the T4 phage particles and/or HOC and/or SOC-fusion peptides of the present invention. These compositions may be antigenic or immunogenic compositions and may be used as vaccines. The compositions may comprise a plurality of recombinant T4 phage particles of the present invention, each comprising a different HOC or SOC fusion peptide. In a specific embodiment, the composition is multivalent vaccine and comprises T4 particles expressing immunogenic or antigenic peptides derived from two or more Helicobacter pylori strains or peptides and/or two or more SCLC peptides. Thus the invention is directed to a method for modulating the growth of a pathogen and/or tumor in an animal in need thereof comprising administering to said animal the composition of the present invention in an amount effective to modulate growth of a pathogen and/or tumor in said animal. The animal may be an avian and a mammal, such as dog, cat, human, cow or pig. The invention is further directed to the use of the particles of the present invention for the manufacture of a medicament for modulating the growth of a pathogen or tumor in an animal.

In yet another embodiment, the invention is directed to a library of the recombinant T4 phage particles and/or HOC and/or SOC fusion peptides of the present invention. As defined herein, a “library” is a mixture of two or more recombinant T4 phage particles or HOC or SOC fusion peptides. The recombinant T4 phage particle may express a ligand, receptor, antibody, antigen and/or immunogen. The T4 particle may further contain a detectable label (e.g., colloid golden, green, red or yellow fluorescent protein, Biotin). A variety of libraries containing the recombinant T4 phage particles of the present invention may be constructed
1) Peptide library, in which the insert is a randomized amino acid (aa) covering all 20 amino acids where the peptides function as ligand, antigen, immunogen.
2) Antibody library. Antibody IgG-Fab/Fv single or double chain domain may be cloned (especially for monoantibody gene engineering library) into the expression system of the present invention where the scFV or dcFV peptide is on T4 phage surface.

These libraries, in one embodiment may be used to detect the presence or absence of a pathogen and/or tumor in a sample comprising incubating the library with said sample and detecting the presence or absence binding of a phage particle in said library to said sample, wherein binding of said a phage particle to said sample indicates the presence of said pathogen and/or tumor. If the library is used for diagnosis kit preparation, pathogen elicited antibody in blood, tissue is detected or alternatively, the pathogen itself may be detected.

### BRIEF DESCRIPTION OF THE FIGURES

FIG. 1 shows the construction of ΦT4ΔSoc (T4 phage Soc expression vector).

FIG. 2A shows the pHOC-mutant vector and FIG. 2B shows the construction of ΦT4ΔHoc (T4 phage HOC expression vector).

FIG. 3 shows ΦT4ΔSoc-ΔHoc (T4 phage Soc-Hoc bipartite expression vector).

FIG. 4A shows a p IN-Soc integration vector, 4B shows a p IN-Hoc integration vector.

### DETAILED DESCRIPTION OF THE INVENTION

In accordance with the present invention, there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

The terms “polynucleotide(s)”, “nucleic acid molecule(s)”, “nucleic acid sequences” and “nucleic acids” will be used interchangeably.

Furthermore, the following terms shall have the definitions set out below.

As defined herein “isolated” refers to material removed from its original environment and is thus altered “by the hand of man” from its natural state.

The amino acid residues described herein are preferred to be in the “L” isomeric form. However, residues in the “D” isomeric form can be substituted for any L-amino acid residue, as long as the desired functional is retained by the polypeptide. NH₂ refers to the free amino group present at the amino terminus of a polypeptide. COOH refers to the free carboxy group present at the carboxy terminus of a polypeptide.

The three letter and one letter abbreviations used for the amino acids are those as normally used in the art, i.e.:

| | Three letter | One letter |
|---|---|---|
| Amino Acid | abbreviation | abbreviation |
| Alanine | Ala | A |
| Cysteine | Cys | C |
| Aspartate | Asp | D |
| Glutamate | Glu | E |
| Phenylalanine | Phe | F |
| Glycine | Gly | G |
| Histidine | His | H |
| Isoleucine | Ile | I |
| Lysine | Lys | K |
| Leucine | Leu | L |
| Methionine | Met | M |
| Asparagine | Asn | N |
| Proline | Pro | P |
| Glutamine | Gln | Q |
| Arginine | Arg | R |
| Serine | Ser | S |
| Threonine | Thr | T |
| Valine | Val | V |
| Tryptophan | Trp | W |
| Tyrosine | Tyr | Y |
| γ-carboxyglutamic acid | Gla | V |

For ease of reference, variants used in the fusion proteins of the invention are described by use of the following nomenclature:
Original amino acid(s): position(s): substituted amino acid(s)
Multiple mutations are separated by plus signs

“Nucleic acid construct” is defined herein, is a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term “expression cassette” when the nucleic acid construct contains all the control sequences required for expression of a coding sequence of the present invention.

The term “coding sequence” is defined herein as a portion of a nucleic acid sequence which directly specifies the amino acid sequence of its protein product. The boundaries of the coding sequence are generally determined by a ribosome binding site (prokaryotes) or by the ATG start codon (eukaryotes) of the first open reading frame at the 5′-end of the mRNA and a transcription terminator sequence located just downstream of the open reading frame at the 3′-end of the mRNA. A coding sequence can include, but is not limited to, DNA, cDNA, and recombinant nucleic acid sequences.

A “heterologous” peptide, protein or nucleic acid sequence of a recombinant T4 phage particle is an identifiable segment of a peptide, protein or nucleic acid within a larger peptide, protein or nucleic acid molecule that is not found in association with the larger molecule in nature.

A “vector” may be any vector (e.g., a plasmid or virus) which can be conveniently subjected to recombinant DNA procedures and can bring about the expression or integration of the nucleic acid sequence.

An “origin of replication” refers to those DNA sequences that participate in DNA synthesis.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

A cell has been “transformed” by exogenous or heterologous nucleic acid when such nucleic acid has been introduced inside the cell. The transforming nucleic acid may or may not be integrated (covalently linked) into chromosomal nucleic acid making up the genome of the cell. In prokaryotes, yeast, and mammalian cells for example, the transforming nucleic acid may be maintained on an episomal element such as a plasmid. With respect to eukaryotic cells, a stably transformed cell is one in which the transforming nucleic acid has become integrated into a chromosome so that it is inherited by daughter cells through chromosome replication. This stability is demonstrated by the ability of the eukaryotic cell to establish cell lines or clones comprised of a population of daughter cells containing the transforming nucleic acid.

It should be appreciated that also within the scope of the present invention are nucleic acid sequences encoding the polypeptide(s) of the present invention, which code for a polypeptide having the same amino acid sequence as the sequences disclosed herein, but which are degenerate to the nucleic acids disclosed herein. By “degenerate to” is meant that a different three-letter codon is used to specify a particular amino acid.

The term “peptide” refers to a polymer of at least four amino acids. It may encompass a protein in its entirety or a portion of the protein having functional activity. In a particular embodiment, the peptide has substantially the same activity of the protein. This term also does not refer to or exclude post-expression modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring.

A nucleic acid molecule is “operatively linked” to an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

As defined herein, a “gene” is the segment of DNA involved in producing a polypeptide chain; it includes regions preceding and following the coding region, as well as intervening sequences (introns) between individual coding segments (exons).

As defined herein, T4 iPII and iPIII are genes encoding T4 internal protein II (IPII) and internal protein III (IPIII) respectively.

As defined herein, an “antigen” is a molecule that stimulates production of antibodies As defined herein, an “immunogen” is a molecule that stimulates immune response, production of antibody related to both T and B cells.

As defined herein, a “ligand” is a substance that binds to a receptor.

As defined herein, a “HOC” protein is a T4 highly antigenic capsid protein and is encoded by the hoc gene.

As defined herein, a “SOC” protein is a T4 small outer capsid protein and is encoded by the soc gene.

As defined herein, a “receptor” is a molecule that receives or responds to a specific substance.

### T4 Expression System

The hoc or soc negative T4 expression system of the present invention may be obtained by homologous recombination of a hoc and/or soc negative phage and a T4 phage particle devoid of T4 lysozyme and/or T4 endonuclease V function, also referred to as a e- and/or denV-T4 phage particle. The expression system may contain variant e and/or denV gene sequences as well as wild type or variant ipII and ipIII gene sequences. These variant sequences may contain substitutions, deletions or insertions resulting in sequences encoding inactive T4 lysozyme and/or T4 endonuclease V.

In the event that the T4 expression system is a hoc negative system, a hoc negative phage is used to obtain said T4 expression system. If the T4 expression system is a soc negative system, a soc negative phage is used. Alternatively, a hoc and soc negative phage may also be used for both hoc and soc T4 expression systems. Such phages may be obtained using procedures known in the art such as by genetic crossing or homologous recombination (see, for example, Homyk and Weil, Virology 61:505-523, Karam, J. D. (ed.), Molecular Biology of Bacteriophage T4. ASM Press, Washington D.C., Black, 1974, Virology 60:166-179; Ren et al., 1997, Gene 195:303-311; Ren et al., 1996, Protein Sci. 5:1833-1843 and Malys et al., 2002, J. Mol. Biol. 319:289-304). The presence or absence of hoc and/or soc gene expression, as noted above, may be determined by PCR using hoc and/or hoc primers or Western Blot analysis using antibodies or antisera to hoc and/or soc (see, for example, Black, 1974, Virology 60:166-179; Ren et al., 1997, Gene 195:303-311; Ren et al., 1996, Protein Sci. 5:1833-1843 and Malys et al., 2002, J. Mol. Biol. 319:289-304).

The T4 phage particle is devoid of T4 lysozyme and/or T4 endonuclease V function and optionally devoid of IPII and IPIII function also referred to as a e⁻ and/or denV⁻, ipII-, ipIII-T4 phage particle may also be obtained using procedures known in the art (see, for example, Black, 1974, Virology 60:166-179, Emrich, 1968, Virology 35:158-165). A particular example of such a particle is Eg326 (S12+, ipI⁻, Alt⁻, e⁻, denV⁻) disclosed in Hong and Black, 1993, Gene 136:193-198.

Recombination procedures are well known in the art and as described in the Examples herein.

The T4 expression system of the present invention may be isolated using methods known in the art. In a particular embodiment, the T4 phage particles may be tested for egg white lysozyme independent and dependent growth. If the resulting phage is e⁻, the phage would only grow in the presence of egg white lysozyme. These particles would be isolated and the presence or absence of expression of soc, hoc would be determined using methods known in the art. These include but are not limited to PCR using ipI, soc or hoc primers or Western Blot analysis using antibodies or antisera to ipI, soc or hoc (see, for example, Black, 1974, Virology 60:166-179; Ren et al., 1997, Gene 195:303-311; Ren et al., 1996, Protein Sci. 5:1833-1843 and Malys et al., 2002, J. Mol. Biol. 319:289-304). The resulting particles containing the recombinant T4 expression system of the present invention may be further isolated using procedures known in the art such as sucrose gradient centrifugation, CsCl gradient centrifguation, glycerol centrifugation (see, for example, Mooney et al., 1987, J. Virol. 61:2828-2834, Ren et al., 1996, Protein Sci. 5:1833-1843).

In the event that the T4 expression system is both soc and hoc negative, the following procedure may be used. Specifically, a hoc⁻ and soc⁻ recombinant T4 phage expression system may undergo homologous recombination using procedures described above and in the examples set forth as well as using procedures known in the art.

### SOC/HOC Fusion Proteins

The invention is further directed to SOC and HOC fusion peptides or proteins. SOC or HOC sequences are bound to heterologous peptides or proteins. As noted above, these peptides or proteins may be a receptor, ligand, pathogen, antigen, immunogen antibody or bio-drugs. In a particular embodiment, the peptide or protein is derived from an Helicobacter pylori antigenic or immunogenic peptide or protein and may encompass the vacuolating cytotoxin A (VacA), urease (urease A and urease B) and/or heat shock protein A (hspA) proteins or peptide fragments thereof having antigenic or immunogenic activity. The peptide or protein may be derived from an HIV antigenic or immunogenic peptide or protein including but not limited to gp160 and/or variant gp41. In a specific embodiment the HIV peptide is a variant gp41 which is resistant to enfuvirtide (Greenberg and Cammack, 2004, J. Antimicrobial Chemotherapy 54:333-340). Such variants include but are not limited to V38A, Q40H, N43D, G36S+L44M, N42T+N43K, N42T+N43S, V38A+N42D, V38A+N42T and V38E+N42S. These variants may be obtained using site specific mutagenesis techniques known in the art. The heterologous peptide or protein may be derived from one or more SCLC immunogens or antigens including but not limited to the CYFRA21-1, NSE, CEA, CRP and/or ProGRP proteins or peptide fragments thereof having antigenic or immunogenic activity.

The SOC protein has the amino acid sequence (Macdonald et al., 1984, Genetics 106:17-27):

(SEQ ID NO:1)

mastrgyvni ktfeqkldgn kkiegkeisv afplysdvhk
isgahyqtfp sekaaystvy eenqrtewia anedlwkvtg

The HOC protein has the following amino acid sequence (Kaliman et al., 1995, Nucl. Acids Res. 18 (14), 4277):

(SEQ ID NO:2)

mdikvhfhdf shvridcees tfhelrdffs feadgyrfnp
rfrygnwdgr irlldynrll pfglvgqikk fcdnfgykaw
idpqinekee lsrkdfdewl skleiysgnk riephwyqkd
avfeglvnrr rilnlptsa

The SOC or HOC peptide may contain conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain. Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, as well as these in reverse. Alternatively, the nucleotide sequence encoding SOC or HOC peptide may contain alterations which produce silent substitutions, additions, or deletions, but do not alter the properties or activities of the encoded polypeptide. Nucleotide variants produced by silent substitutions due to the degeneracy of the genetic code are preferred.

The fusion protein or peptide may be obtained using recombinant DNA methods. For example, a nucleic acid sequence encoding a heterologous peptide may be inserted into a vector containing nucleic acid sequences encoding the HOC or HOC peptide or SOC or SOC peptide (for example, see FIGS. 4A and 4B). In a particular embodiment, the HOC or SOC sequence is obtained by PCR and inserted, for example, into a pET vector, or other protein expression vector capable of expressing a fusion protein. In a more particular embodiment, the nucleic acid sequence encoding HOC is shown below in SEQ ID NO:3:

| | |
|---|---|
| 1 | ATGACTTTTA CAGTTGATAT AACTCCTAAA ACACCTACAG GGGTTATTGA |
| 51 | TGAAACTAAG CAGTTTACTG CTACACCCAG TGGTCAAACT GGAGGCGGAA |
| 101 | CTATTACATA TGCTTGGAGC GTAGATAATG TTCCACAAGA TGGAGCTGAA |
| 151 | GCAACTTTTA GTTATGTACT AAAAGGACCT GCCGGTCAAA AGACTATTAA |
| 201 | AGTAGTTGCA ACAAATACAC TTTCTGAAGG AGGCCCGGAA ACGGCTGAAG |
| 251 | CGACAACAAC TATCACAGTT AAAAATAAGA CACAGACGAC TACCTTAGCC |
| 301 | GTAACTCCTG CTAGTCCTGC GGCTGGAGTG ATTGGAACCC CAGTTCAATT |
| 351 | TACTGCTGCC TTAGCTTCTC AACCTGATGG AGCATCTGCT ACGTATCAGT |
| 401 | GGTATGTAGA TGATTCACAA GTTGGTGGAG AAACTAACTC TACATTTAGC |
| 451 | TATACTCCAA CTACAAGTGG AGTAAAAAGA ATTAAATGCG TAGCCCAAGT |
| 501 | AACCGCGACA GATTATGATG CACTAAGCGT TACTTCTAAT GAAGTATCAT |
| 551 | TAACGGTTAA TAAGAAGACA ATGAATCCAC AGGTTACATT GACTCCTCCT |
| 601 | TCTATTAATG TTCAGCAAGA TGCTTCGGCT ACATTTACGG CTAATGTTAC |
| 651 | GGGTGCTCCA GAAGAAGCAC AAATTACTTA CTCATGGAAG AAAGATTCTT |
| 701 | CTCCTGTAGA AGGGTCAACT AACGTATATA CTGTCGATAC CTCATCTGTT |
| 751 | GGAAGTCAAA CTATTGAAGT TACTGCAACT GTTACTGCTG CAGATTATAA |
| 801 | CCCTGTAACC GTTACCAAAA CTGGTAATGT AACAGTCACG GCTAAAGTTG |
| 851 | CTCCAGAACC AGAAGGTGAA TTACCTTATG TTCATCCTCT TCCACACCGT |
| 901 | AGCTCAGCTT ACATCTGGTG CGGTTGGTGG GTTATGGATG AAATCCAAAA |
| 951 | AATGACCGAA GAAGGTAAAG ATTGGAAAAC TGACGACCCA GATAGTAAAT |
| 1001 | ATTACCTGCA TCGTTACACT CTCCAGAAGA TGATGAAAGA CTATCCAGAA |
| 1051 | GTTGATGTCC AAGAATCGCG TAATGGATAC ATCATTCATA AAACTGCTTT |
| 1101 | AGAAACTGGT ATCATCTATA CCTATCCATA ATCATAAGGG GCTTCGGCCC |
| 1151 | CTTTCTTCAT TTTGAAAGCA CACAAAACAC AATCAGAAAA TGATGTATAT |
| 1201 | AATGGCACCA ACTCGATAAC ATGA |

In one embodiment, the nucleic acid sequence encoding a heterologous peptide is immediately adjacent to the sequence encoding HOC or SOC or HOC or SOC functional peptide. In another embodiment, the two sequences are separated by a linker or spacer sequence. In a specific embodiment, the linker or spacer sequence may comprise a cleavage site (e.g., trypsin cleavage site). Mutation of the nucleic acid sequence encoding the heterologous peptide may be accomplished using site specific mutagenesis procedures known in the art.

The fusion protein may be expressed in a eukaryotic or prokaryotic cell. In a preferred embodiment, the fusion protein would be expressed in a prokaryotic cell (e.g., E. coli) and would be isolated and purified using procedures known in the art, such as HPLC and column chromatography.

### Recombinant T4 Phage Particles

The recombinant T4 phage particles of the present invention contain or express a SOC and/or HOC fusion peptide. In a particular embodiment, the T4 phage particle is iPII and iPIII negative as well as e and/or denV negative, also referred to as ipII⁻, ipIII⁻, e⁻ and/or denV⁻. The recombinant T4 phage particle of the present invention may be obtained by first transforming the prokaryotic cell, preferably E. coli with a vector comprising a nucleic acid sequence encoding the hoc or soc fusion protein. If the prokaryotic cell is transformed with a HOC fusion plasmid, the transformed cell is subsequently infected with a hoc⁻ recombinant expression system of the present invention. If the prokaryotic cell is transformed with a SOC fusion plasmid, the transformed cell is subsequently infected with a soc⁻ recombinant expression system of the present invention. If the prokaryotic cell is transformed with a HOC and SOC fusion plasmid, the transformed cell is subsequently infected with a hoc⁻soc⁻ recombinant expression system of the present invention.

Plaques are isolated and checked for integration of desired heterologous nucleic acid sequences using methods known in the art, e.g., PCR primers.

### Vaccines

The recombinant T4 phage particles of the present invention may be used to formulate compositions. In a particular embodiment, the compositions of the present invention may be immunogenic or antigenic compositions and may thus be used in vaccine formulations. In a particular embodiment, the compositions and particularly the vaccine formulations of the present invention may comprise recombinant T4 phage particles expressing one or more HOC- and/or SOC-: Helicobacter pylori, HIV and/or SCLC fusion peptides.

In a more particular embodiment, the compositions may be multivalent vaccines. For example, one composition may comprise one or more Helicobacter pylori antigenic or immunogenic peptides or in a particular embodiment, from two or more strains; another composition may comprise multiple SCLC antigenic or immunogenic proteins.

In a more particular embodiment, the composition of the present invention may comprise the recombinant T4 phage particles of the present invention that express a HOC- and/or SOC- The vaccine delivery systems of the present invention can be prepared in a physiologically acceptable formulation, such as in a pharmaceutically acceptable carrier, using known techniques. For example, the recombinant T4 phage particles of the present invention may be combined with a pharmaceutically acceptable excipient to form an immunogenic composition. Alternatively, these recombinant T4 phage particles may be administered in a vehicle having specificity for a target site, such as a tumor or infection.

The vaccine delivery vehicles of the present invention may be administered in the form of a solid, liquid or aerosol. Examples of solid compositions include but are not limited to pills, creams, and implantable dosage units. Pills may be administered orally. Therapeutic creams may be administered topically. Implantable dosage units may be administered locally, for example, at a tumor site, or may be implanted for systematic release of the therapeutic composition, for example, subcutaneously. Examples of liquid compositions include but are not limited to formulations adapted for injection intramuscularly, subcutaneously, intravenously, intra-arterially, and formulations for topical and intraocular administration. Examples of aerosol formulations include inhaler formulations for administration to the lungs.

The compositions may be administered by standard routes of administration. In general, the composition may be administered by topical, oral, rectal, nasal or parenteral (for example, intravenous, subcutaneous, or intramuscular) routes. In addition, the composition may be incorporated into sustained release matrices such as biodegradable polymers, the polymers being implanted in the vicinity of where delivery is desired, for example, at the site of a tumor. The method includes administration of a single dose, administration of repeated doses at predetermined time intervals, and sustained administration for a predetermined period of time.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly (ortho) esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The dosage of the vaccine composition made according to the present invention depends on the species, breed, age, size, vaccination history, and health status of the animal to be vaccinated. Other factors like antigen concentration, additional vaccine components, and route of administration (i.e., subcutaneous, intradermal, oral, intramuscular or intravenous administration) will also impact the effective dosage. The dosage of vaccine to administer is easily determinable based on the antigen concentration of the vaccine, the route of administration, and the age and condition of the animal to be vaccinated. Each batch of antigen may be individually calibrated. Alternatively, trials of different dosages, as well as LD₅₀ studies and other screening procedures can be used to determine effective dosage for the immunogenic or antigenic composition of the present invention. From the examples presented below, it will be readily apparent what approximate dosage and what approximate volume would be appropriate for using the vaccine composition described herein. The critical factor is that the dosage provides at least a partial protective effect against natural infection, as evidenced by a reduction in the mortality and morbidity associated with natural infection. The appropriate volume is likewise easily ascertained by one of ordinary skill in the art. For example, in avian species the volume of a dose may be from about 0.1 ml to about 0.5 ml and, advantageously, from about 0.3 ml to about 0.5 ml. For feline, canine and equine species, the volume of a dose may be from about 0.2 ml to about 3.0 ml, advantageously from about 0.3 ml to about 2.0 ml, and more advantageously, from about 0.5 ml to about 1.0 ml. For bovine and porcine species, the volume of dose may be from about 0.2 ml to about 5.0 ml, advantageously from about 0.3 ml to about 3.0 ml, and more advantageously from 0.5 ml to about 2.0 ml.

Repeated vaccinations may be preferable at periodic time intervals to enhance the immune response initially or when a long period of time has elapsed since the last dose. In one embodiment of the present invention, the vaccine composition is administered as a parenteral injection (i.e., subcutaneously, intradermally, or intramuscularly. The composition may be administered as one dose or, in alternate embodiments, administered in repeated doses of from about two to about five doses given at intervals of about two to about six weeks, preferably from about two to about five weeks. However, one of skill in the art will recognize that the number of doses and the time interval between vaccinations depends on a number of factors including, but not limited to, the age of the animal vaccinated; the condition of the animal; the route of immunization; amount of antigen available per dose; and the like. For initial vaccination, the period will generally be longer than a week and preferably will be between about two to about five weeks. For previously vaccinated animals, a booster vaccination, before or during pregnancy, at about an annual interval may be performed.

### Diagnostic Methods and Kits

The particles of the present invention may be used for diagnostic purposes. In a specific embodiment, the expressed HOC or SOC fusion peptides are linked to detectable label. In a most specific embodiment, the detectable label is a fluorescent or luminescent protein such as green fluorescent protein, golden, or yellow fluorescent protein. The kit may comprise a library of the recombinant T4 phage particles of the present invention. These libraries comprise a multiplicity of the T4 phage particles of the present invention. These phage particles may express nucleic acid sequences encoding HOC and/or SOC-heterologous antigenic and immunogenic fusion peptides as well as antibodies.

In one embodiment, the kit of the present invention may contain a library of recombinant T4 phage particles expressing a plurality of Helicobacter pylori peptides in one embodiment from various strains. These peptides would be capable of binding to antibodies to Helicobacter pylori since they are antigenic or immunogenic. The binding of any of the peptides with a patient sample would indicate Helicobacter pylori infection. Either the T4 phage particles and/or patient sample may be labeled with a detectable label.

In another embodiment, the kits of the present invention may comprise a library of recombinant T4 phage particles expressing HOC and/or SOC-small cell lung carcinoma (SCLC) antigenic or immunogenic fusion peptides which include but are not limited to CYFRA21-1, NSE, CEA, CRP and/or ProGRP proteins.

In yet another embodiment, the kits of the present invention may comprise a library of recombinant T4 phage particles express HOC and/or SOC HIV antigenic or immunogenic fusion proteins which include but are not limited to gp 160 and/or variant gp41. The variant gp41 peptides or proteins include but are not limited to V38A, Q40H, N43D, G36S+L44M, N42T+N43K, N42T+N43S, V38A+N42D, V38A+N42T and V38E+N42S.

### High Throughput Screening (FACS 3-D Platform), Biopanning (Solid Phase 2-D Platform)

The invention is further directed to methods and kits for identifying ligands binding to particular receptors or particularly, high throughput screening of possible drug or vaccine candidates. In a particular embodiment, a library of the recombinant T4 phage particles of the present invention are contacted with a target substance, such as an antigen or immunogen of interest. In a particular embodiment, the target substance may be a protein receptor. This receptor may actually be expressed on a recombinant T4 phage particle as a SOC and/or HOC fusion peptide or Phage Expression, packaging, processing system (PEPP) (Biotechniques 1998, 25:1008-1012) and may also contain a detectable label, such as a fluorescent protein. In a particular embodiment, the recombinant T4 phage particles and/or antigen or immunogen are labeled with a detectable label. The presence or absence of binding of the recombinant phage particles to a target substance is subsequently detected using methods known in the art, e.g., FACS 3-D platform or by a solid phase 2-D platform.

In another particular embodiment, a recombinant T4 phage particle containing a receptor and label expressed as a HOC or SOC fusion protein is contacted with a peptide library. The presence or absence of binding of peptides in the library to said recombinant T4 phage particle may be detected using methods known in the art.

### EXAMPLES

### Example 1

### High Efficiency T4 Bacteriophage Surface Protein Expression System: Vectors and Construction

The construction of three systems are described:
1.) ΦμTΔSoc+p IN-Soc (T4 phage Soc site expression system)
2.) ΦT4ΔHoc+p IN-Hoc (T4 phage Hoc site expression system)
3.) ΦT4ΔSoc ΔHoc+p IN-Soc-Hoc (T4 phage Soc-Hoc bipartite sites expression system): Recombination between ΦT4ΔSoc and ΦT4ΔHoc to create the double deletion mutant without damaging the other genes functions.

### ΦT4ΔSoc+p IN-Soc (T4 Phage Soc Site Expression System)

The following procedure was used in obtaining the above T4 phage Soc site expression system This expression system is more efficient than previously published systems and contains several unique endonuclease sites (SmaI, XbaI, SalI, NcoI etc.) at the EcoRI site to facilitate heterologous gene insertion;

The T4 phage, T4-Δ9.8 Soc was crossed with T4 phage eG326 in one host E. coli CR63 using the homologous recombination procedure described above. T4-Δ9.8Soc was described in Homyk et al., 1974, Virology 61:505-523. This phage T4-Δ9.8 Soc contains approximately a 9.8 kb deletion between T4 genes 39 and 56. T4 phage strain eG326 was described in Hong Y R, Black L W. 1993, Gene 136:193-198.

E. coli CR63 in CM medium (per liter contains: 10 g Tryptone, 5 g NaCl, 50 ml Tris.CL, pH 7.5, 10 ml 25% Na— Citrate), at 0.2 (OD600), was inoculated with both phage T4-Δ9.8Soc and eG326 at MOI (multiplicity of infection) 0.5 as described in Ren et al., 1996, Protein Science 5: 1833-1843. The culture was incubated at 35° C. overnight, spread on lysozyme 200 ug/ml containing and no lysozyme CM plates. The plates were incubated at 37° C. overnight. The PFU number on the lysozyme plate was double the amount than on the lysozyme deficient plate. All PFUs from the lysozyme containing plate were collected, inoculated into E. coli CR63 again and the process was repeated. The PFU number was much larger in lysozyme containing plates than on lysozyme deficient plates. Phage particles from second round lysozyme plate were collected and the process was repeated a third time. 20 single plaques were selected at random from the lysozyme plate and subjected to PCR analysis for Soc gene deletion and IPII-IPIII deletion.

Anti-Soc antibody was used as a probe to screen the 9.4 kDa Soc protein band deletion by Western Blotting. All plaques found to contain the SOC deletion by PCR analysis was also found to be SOC negative via Western Blot analysis. No Soc positive protein bands appeared. Some were IPII-IPIII negative; others were not.

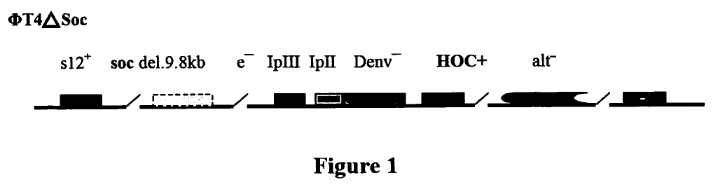

The final vector is shown in FIG. 1.

### ΦT4ΔHoc+p IN-Hoc (T4 Phage Hoc Site Expression System)

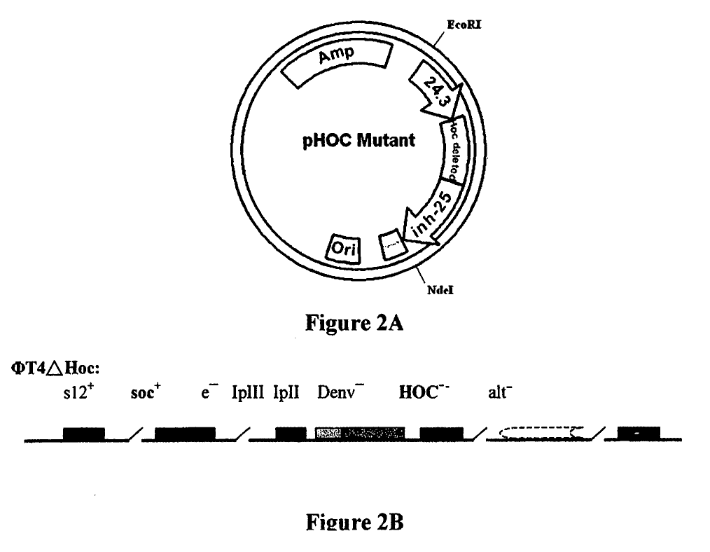

First, PΔHoc376aa is obtained and is shown in FIG. 2A. This contains approximately a 376 aa (1128 bp) deletion between genes 24 and inh-25. PΔHoc376aa was constructed as follows. The Soc-V3 fragment was removed from the plasmid vector, pE V-3 (see Ren et al., 1996, Protein Science 5:1833-1843) using restriction enzymes NdeI and EcoRI as vector. The fragment to be inserted into pE was obtained by ligating two PCR fragments together using wild T4 phage DNA as template. For the first PCR, one primer was located at far hoc gene end, the 186 bp position, the opposite primer extended around ˜500 bp away in T4 24 gene area. For the second PCR, one primer was located at far hoc gene end, at 156 bp position; the opposite primer extended around ˜500 bp away in T4 inh-25 gene area. These two PCR pieces were ligated together as ˜1 kb insert into pE and transformed into E. coli. CR63. A homologous recombination reaction was undertaken by infecting transformed E. coli. CR63 with wild T4 phage. The conditions were analogous to those described above for T4 phage soc site expression system. E. coli CR63 was grown in CM medium, while OD600 to around 0.2, inoculated with T4 wild phage at MOI 0.5, and cultured at 35° C. overnight. Plaques were spread on common LB plates and grown at 37° C. overnight. All plaques were collected and the process was repeated two more times. Isolated plaques were screened for the Hoc deletion via PCR and Western Blot analysis using an anti-Hoc antibody.

The final vector is shown in FIG. 2B.

### ΦT4ΔSoc-ΔHoc+p IN-Soc-Hoc (T4 Phage Soc-Hoc Bipartite Sites Expression System)

The T4 phage Soc-Hoc bipartite sites expression vector was obtained by homologous recombination between ΦT4ΔSoc and Φ4T4ΔHoc in E. coli CR63. The first step involved a homologous recombination reaction between ΦT4ΔSoc and ΦT4ΔHoc. 9.

OD 600 E. coli CR63 was infected with T4 phage mutants ΦT4ΔSoc and ΦT4ΔHoc together and incubated as described above. E. coli CR63 was grown in CM medium (OD600 to around 0.2), inoculated with phage ΦT4ΔSoc and ΦT4ΔHoc both at MOI 0.5, and cultured at 35° C. overnight, spread on common LB plates, and grown at 37° C. overnight. All plaques were collected, and the process was repeated twice.

20 resulting phage particles were collected and screened for Soc and Hoc deletions by PCR and by Western Blotting using anti-Hoc and anti-Soc antibodies.

A couple of CM plates were prepared. One was lysozyme containing and another was lysozyme free. A plaque containing Soc and Hoc deletions, and lysozyme dependence was isolated.

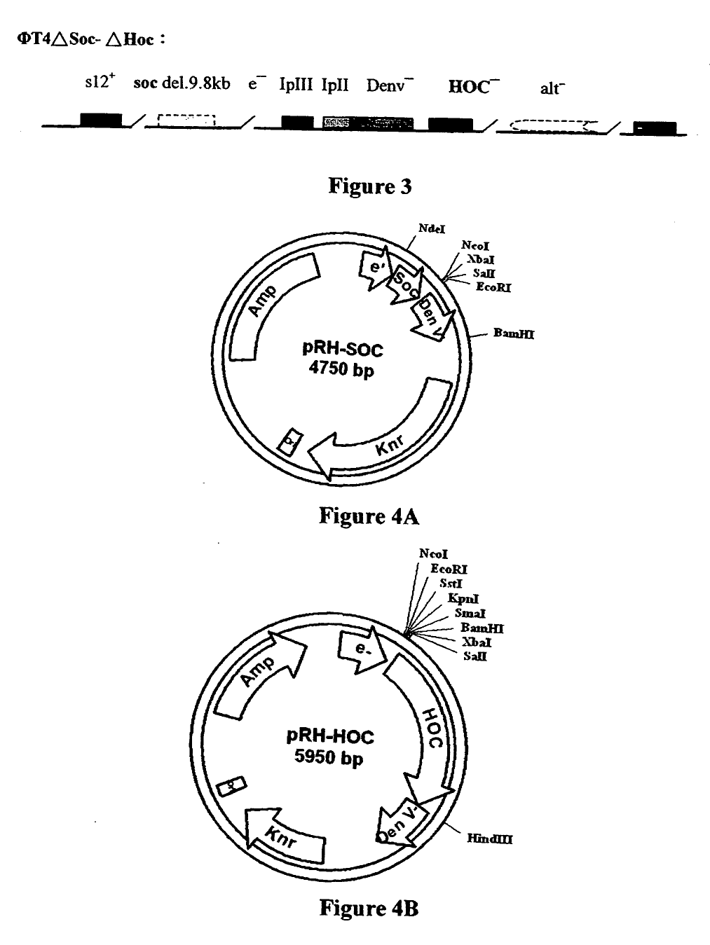

The final vector is shown in FIG. 3.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties.

What is claimed is:

1. A recombinant T4 phage particle comprising a SOC and/or HOC fusion peptide, wherein said phage particle has inactive T4 endonuclease V and/or T4 lysozyme function wherein said fusion peptide comprises SOC and/or HOC bound to one or more heterologous peptides derived from one or more Helicobacter pylori immunogenic or antigenic peptides, HIV gp160, and variant gp41 and/or small cell lung carcinoma (SCLC) peptides or proteins.

2. The recombinant T4 phage particle according to claim 1, wherein said fusion peptide comprises SOC and/or HOC bound to peptide derived from a SCLC immunogenic or antigenic peptide.

3. The recombinant T4 phage particle according to claim 1 wherein said fusion peptide comprises SOC and/or HOC bound to one or more heterologous peptides derived from a SCLC antigenic or immunogenic peptide selected from the group consisting of cytokeratin 19 (CYFRA21-1), neuronal specific enolase (NSE), carcinoembryonic antigen (CEA), C-reactive protein (CRP) and pro-gastrin releasing peptide (ProGRP) protein.

4. The recombinant T4 phage particle according to claim 1, wherein said heterologous peptide is a toxin, antigen or immunogen derived from one or more Helicobacter pylori strains.

5. The recombinant T4 phage particle according to claim 1, wherein said particle comprises one or more of the heterologous peptides derived from Helicobacter pylori.

6. The recombinant T4 phage particle according to claim 1, wherein said particle comprises a HOC- and/or SOC bound to one or more heterologous antigenic or immunogenic peptides derived from Helicobacter pylori vacuolating cytotoxin A (VacA), urease (urease A and urease B) and/or heat shock protein A (hspA) proteins.

7. The recombinant T4 phage particle according to claim 1 wherein said particle comprises a HOC and/or SOC bound to one or more heterologous peptides derived from HIV gp160, and/or variant gp41 peptide or protein.

8. The recombinant T4 phage particle of claim 7, wherein said variant gp41 peptide or protein is selected from the group consisting of V38A, Q40H, N43D, G36S+L44M, N42T+N43K, N42T+N43S, V38A+N42D, V38A+N42T and V38E+N42S

9. An isolated fusion peptide comprising HOC or SOC bound to a heterologous peptide derived from one or more Helicobacter pylori, HIV gp160, and/or variant gp41 and/or SCLC immunogenic or antigenic peptides.

10. An isolated nucleic acid molecule encoding the fusion peptide of claim 9.

11. A nucleic acid construct comprising the isolated nucleic acid molecule of claim 10.

12. A recombinant T4 phage expression system comprising the isolated nucleic acid molecule of claim 10.

13. The recombinant T4 phage expression system of claim 12, wherein said expression system is a hoc⁻ and/or soc⁻, ipII⁺ and/or ipIII⁺ T4 phage expression system, wherein said expression system is additionally e⁻, and/or denV³¹ .

15. A prokaryotic cell comprising the isolated nucleic acid molecule of claim 10.

16. A prokaryotic cell comprising the nucleic acid construct of claim 11.

17. A prokaryotic cell comprising the recombinant T4 phage particle of claim 1.

18. A prokaryotic cell comprising the recombinant T4 phage expression system of claim 12.

19. A method for obtaining a HOC and/or SOC fusion protein comprising
(a) culturing the host prokaryotic cell of claim 15 and
(b) isolating the HOC and/or SOC protein.

20. A composition comprising one or more recombinant T4 phage particles of claim 1 and a carrier.

21. A method of modulating growth of Helicobacter pylori and/or SCLC in an animal comprising administering to said animal in need thereof the T4 phage particle of claim 1 in an amount effective to modulate growth of a pathogen and/or tumor in said animal.

22. A library of recombinant T4 phage particles of claim 1.

23. A method of detecting the presence or absence of a Helicobacter pylori or SCLC in a sample comprising incubating the library of claim 22 with said sample and detecting the presence or absence of binding of a phage particle in said library to said sample.

24. A kit comprising the library of claim 22.


SEQUENCE LISTING (11 sequences)

SEQ ID NO: 1 - DNA, 21 bp, Artificial Sequence
  Description of Artificial Sequence Synthetic primer
ctcaacgcagaatggaaagca

SEQ ID NO: 2 - DNA, 21 bp, Artificial Sequence
  Description of Artificial Sequence Synthetic primer
ggtcgaagttcagaagctgtt

SEQ ID NO: 3 - DNA, 32 bp, Artificial Sequence
  Description of Artificial Sequence Synthetic primer
cggaattcgtcaaaccagtactgatactcgcc

SEQ ID NO: 4 - protein, 80 aa, Enterobacteria phage T4
MASTRGYVNIKTFEQKLDGNKKIEGKEISVAFPLYSDVHKISGAHYQTFPSEKAAYSTVYEENQRTEWIAANEDLWKVTG

SEQ ID NO: 5 - protein, 139 aa, Enterobacteria phage T4
MDIKVHFHDFSHVRIDCEESTFHELRDFFSFEADGYRFNPRFRYGNWDGRIRLLDYNRLLPFGLVGQIKKFCDNFGYKAWIDPQINEKEELSRKDFDEWLSKLEIYSGNKRIEPHWYQKDAVFEGLVNRRRILNLPTSA

SEQ ID NO: 6 - DNA, 1224 bp, Enterobacteria phage T4
atgacttttacagttgatataactcctaaaacacctacaggggttattgatgaaactaagcagtttactgctacacccagtggtcaaactggaggcggaactattacatatgcttggagcgtagataatgttccacaagatggagctgaagcaacttttagttatgtactaaaaggacctgccggtcaaaagactattaaagtagttgcaacaaatacactttctgaaggaggcccggaaacggctgaagcgacaacaactatcacagttaaaaataagacacagacgactaccttagccgtaactcctgctagtcctgcggctggagtgattggaaccccagttcaatttactgctgccttagcttctcaacctgatggagcatctgctacgtatcagtggtatgtagatgattcacaagttggtggagaaactaactctacatttagctatactccaactacaagtggagtaaaaagaattaaatgcgtagcccaagtaaccgcgacagattatgatgcactaagcgttacttctaatgaagtatcattaacggttaataagaagacaatgaatccacaggttacattgactcctccttctattaatgttcagcaagatgcttcggctacatttacggctaatgttacgggtgctccagaagaagcacaaattacttactcatggaagaaagattcttctcctgtagaagggtcaactaacgtatatactgtcgatacctcatctgttggaagtcaaactattgaagttactgcaactgttactgctgcagattataaccctgtaaccgttaccaaaactggtaatgtaacagtcacggctaaagttgctccagaaccagaaggtgaattaccttatgttcatcctcttccacaccgtagctcagcttacatctggtgcggttggtgggttatggatgaaatccaaaaaatgaccgaagaaggtaaagattggaaaactgacgacccagatagtaaatattacctgcatcgttacactctccagaagatgatgaaagactatccagaagttgatgtccaagaatcgcgtaatggatacatcattcataaaactgctttagaaactggtatcatctatacctatccataatcataaggggcttcggcccctttcttcattttgaaagcacacaaaacacaatcagaaaatgatgtatataatggcaccaactcgataacatga

SEQ ID NO: 7 - DNA, 25 bp, Artificial Sequence
  Description of Artificial Sequence Synthetic primer
gaatcatatggctagtactcgcggt

SEQ ID NO: 8 - DNA, 38 bp, Artificial Sequence
  Description of Artificial Sequence Synthetic primer
caggctaagcgcaagcaaccagttactttccacaaatc

SEQ ID NO: 9 - DNA, 38 bp, Artificial Sequence
  Description of Artificial Sequence Synthetic primer
aactggttgcttgcgcttagcctgcaaggaagattacc

SEQ ID NO: 10 - DNA, 28 bp, Artificial Sequence
  Description of Artificial Sequence Synthetic primer
cggaattcaaattgggcagacaaggtag

SEQ ID NO: 11 - DNA, 33 bp, Artificial Sequence
  Description of Artificial Sequence Synthetic primer
accatggtacgcttagcctgcaaggaagattac